(19) United States
(12) Patent Application Publication
(10) Pub. No.: US 2012/0078079 A1
(43) Pub. Date: Mar. 29, 2012

(54) USE OF THE HEART RATE VARIABILITY CHANGE TO CORRELATE MAGNETIC FIELD CHANGES WITH PHYSIOLOGICAL SENSITIVITY AND METHOD THEREFOR

(76) Inventor: Elisabeth Plank, Salzburg (AT)

(21) Appl. No.: 13/262,114

(22) PCT Filed: Mar. 30, 2010
(86) PCT No.: PCT/EP2010/054191

§ 371 Date: Dec. 9, 2011

(30) Foreign Application Priority Data
Apr. 2, 2009 (DE) 10 2009 002 134.5

(51) Int. Cl. A61B 5/05

(52) U.S. Cl. 600/407

(57) ABSTRACT

The invention relates to the use of a device to analyze heart rate variability to determine changes of the physiological state of a test subject due to a change of a magnetic field acting on the test subject, comprising the analysis of the heart rate variability of the test subject before and after the change of the acting magnetic field. A corresponding method comprises the steps of: analyzing the heart rate variability of the test subject; making changes to the magnetic field acting on the test subject; analyzing the heart rate variability of the test subject again; and evaluating a change of the physiological state of the test subject based on the change of the heart rate variability between the measurement before and after the magnetic field change.

In recent years, the study of the effects especially of magnetic fields on organisms has come into prominence as a further focus in the field of the study of effects of so-called electrosmog on animal organisms, alongside studies on the effects of electric fields, in particular high-frequency electric fields. There are various findings that suggest a correlation between the physiological state of organisms and the magnetic fields acting on them. These studies are at an early stage such that, alongside the clearly apparent correlations between magnetic field effects and physiological states of organisms, there are currently only attempts to explain the potential causalities.

Without wanting to be bound to a specific theory, for example, a relationship between magnetic fields and the rhythmic as well as other chronometric controls of the organism has been suspected which could potentially be related to the natural magnetic field of the Earth in the ultra-low-frequency (ULF) frequency range up to 15 Hz. It is also known from scientific literature that animal or human organisms in this frequency range have a special sensitivity even at very low power ranges of the radiation.

In addition to well-studied thermal effects of the action of electromagnetic radiation on organisms which are characterised by heating of body tissue in the event of action of electromagnetic radiation with higher intensity on organisms, further non-thermal effects on the organism have also been studied, as a result of the above considerations in relation to the direct action of magnetic fields on the control of the rhythm of organisms.

Non-thermal effects can occur if the power is low to very low. These effects are not based on heating of tissue, but rather lead, by means of various other mechanisms, to changes in the body. Athermal effects can have a negative effect in terms of stress on the body, functional changes of cells, organs or cellular processes and cell rhythm through to organic illnesses or damage to DNA. Specific frequencies, however, also have positive effects and are used e.g. in medical therapy.

The electromagnetic field in the ultra-low-frequency range up to 15 Hz exerts a central and determining control function on biological processes in cells, plants, animals and humans.

The manner of this influence—whether beneficial or pathogenic—is, on the one hand, dependent on the type and power of incident electromagnetic radiation, but is, on the other hand, dependent to a greater extent on the homogeneity of the above-mentioned ULF field.

A low homogeneity of the ULF field has in a variety of ways a disturbing influence on the biological processes of organisms. It represents, particularly in the case of longer action, a stressing situation for living things and can lead to a wide range of symptoms through to obvious illnesses.

This overriding controlling instance of the ULF field can be proved at any time in short-term studies. Long-term studies likewise showed that the influence of magnetic fields is of acute and constant importance.

However, the subjective perception of such phenomena is difficult to record. People can become accustomed to a reduced general and regulation condition over a long period of time and therefore only perceive a further deterioration as noticeable, while they refer to the normal poor condition as “I′m fine”.

Only a measurement which is independent of the “perception” of the person could objectivise the actual condition and thus draw attention to chronic-lingering stresses.

Living organisms can namely in the case of a longer term presence of stimuli of any type become used to these such that permanently present stimuli are no longer consciously perceived. Longer lasting noise pollution is thus often no longer consciously “heard”, but still places a stress on the vegetative nervous system. The same observation can also be made in the case of stressing electromagnetic fields.

In the range of electromagnetic radiation, extremely long-acting stimuli can also trigger overloading reactions in the sense of a hypersensitivity or “allergy” to the corresponding initiator. This effect known from aliergology can also occur in the context of electrosmog. If a person comes into contact with the corresponding frequency, even highly acute conditions can be triggered (localised or generalised cramps, pains, numbness, tinnitus, dizziness, headaches, sleepiness, etc.)

This circumstance has already been known for a long time in the field of stressing with electromagnetic fields and is described in the relevant literature. The frequency of electrosensitivity in the general population is a few percent depending on the source, but is a problem which is increasing from year to year.

It became necessary to supplement the physical measurement of (biologically relevant) magnetic field influences with a biological or physiological measurement method which is suitable for the mapping of direct influences on ULF magnetic fields on test subjects such as humans.

The object of the present invention was thus to provide an objectivised approach to studying the influence of magnetic fields and—in particular as a result of the omnipresence of magnetic fields in the environment—of the influence of specific changes of magnetic fields on organisms, in particular humans.

It has been surprisingly shown that such an objectivisable method is available for use by measuring the so-called heart rate variability. Heart rate variability determination is a recognised method for an objectivised evaluation of the physiological condition of a person and has already been used at an earlier stage for testing medicament effects, stress in the workplace as well as cardiovascular health. Historically, heart rate variability analysis is based originally on the observation that, in the case of heart attack patients or patients with cardiac insufficiency and thus a high risk of a heart attack, the heart rate variability is impaired and the heart beats almost in a manner of an emergency programme in a more monotonous and less variable manner than in the case of healthy people.

However, the method for analysing heart rate variability is hitherto not known for ascertaining a relationship between changes in magnetic field and the physiological condition of a person.

Therefore, in one aspect, the invention is directed at the use of a device for analysing heart rate variability in order to determine changes in the physiological condition of a test subject due to a change in a magnetic field acting on the test subject, comprising the analysis of the heart rate variability of the test subject in each case before and after the change in the acting magnetic field.

In a further aspect, the invention is directed at a method for determining changes in the physiological condition of a test subject on the basis of his heart rate variability due to a change in a magnetic field acting on the test subject, comprising the steps:
analysing the heart rate variability of the test subject;
making changes to the magnetic field acting on the test subject;
re-analysing the heart rate variability of the test subject, and
evaluating a change in the physiological condition of the test subject on the basis of the change in the heart rate variability between the measurement before and after the change in magnetic field.

The test subject is preferably a mammal and particularly preferably a human. Other species can, however, also be tested in so far as they have a corresponding regulatory system that varies the heart rate.

There are various possibilities for the procedure over time. The necessary individual steps can thus be carried out in immediate succession. This shows the direct effects of a change in the magnetic field acting on the test subject.

The renewed analysis of the heart rate variability can, however, only be carried out 1 to 30 days after the change in the magnetic field so that longer term influences as a result of the change in the magnetic field can also be detected which do not come about immediately after the change. Of course, both measurements can be combined, i.e. an immediate measurement and a subsequent control measurement can be carried out.

The analysis of the heart rate variability preferably comprises several steps:
measuring the pulse of the test subject by means of an ECG;
determining the heart rate variability from the pulse; and
evaluating the heart rate variability in terms of the physiological condition of the test subject.

These steps are familiar to experts in the field of heart rate analysis and in particular corresponding evaluation programs or diagrams are commercially available.

The analysis can furthermore include the generation of a regulation value (R value) which numerically reflects the quality of the physiological condition of the test subject over the period of measurement. The R value, which is described in detail further below, is an accepted measure for simple evaluation of the heart rate variability in a single FIGURE.

In this case, it is preferably assumed that a change in the physiological condition of the test subject exists in the event of a change by more than 10%, preferably by more than 20% in the case of the R value.

In a further preferred embodiment, the invention further comprises the use of a device for measuring the magnetic field acting on the test subject, for correlation of the change in the magnetic field with the change in the physiological condition of the test subject.

Such a measurement of the magnetic field can be carried out in a frequency range from 0 to 15 Hz of oscillating or fluctuating magnetic fields. Frequencies in the ultra-low-frequency spectrum are currently suspected of having significant effects on living organisms, including humans and are therefore one of the key focuses of interest.

The frequency range from 0-15 Hz is particularly preferably used for measurements in order to prevent interference with influences of technical frequencies (beginning with 16⅔ Hz in the case of railway current). Other frequency ranges including unchangeable magnetic field can, however, also be detected.

The measurement is preferably carried out on one plane at a spatial position at which the test subject spends at least some time during analysis of the heart rate variability, the measurement having the following steps.
definition of a surface, which lies on the plane, of a predefined size;
specifying a pattern of measurement points on the surface;
measuring the magnetic field strength at the measurement points; and
determining the magnetic field and the magnetic field homogeneity across the measured surface.

In this case, the surface should be dimensioned such that it can detect the key influences of the magnetic field on the test subject. Depending on the question and further scientific knowledge with regard to the specificity of the action of the magnetic field on organisms, it is also conceivable that the orientation of the measurement plane (e.g. horizontal or vertical) also plays a role and is adapted in accordance with the question.

The change in the magnetic field can be carried out in a simple manner and with foreseeable results where only a single magnetic field source dominates (wherein the Earth's magnetic field can be regarded as given). However, in the case of use, in the attempt to eliminate disorders in people which could be due to magnetic fields, several magnetic field sources typically occur however, such as electronic devices, metal objects which resonate with an oscillating magnetic field, etc. so that there are several approaches for a change in the magnetic field. Therefore, a change which is made in the magnetic field can potentially not lead to the desired result, i.e. a significant change in the relevant parameters of the heart rate variability. In such cases, it may be expedient to repeat the method several times, wherein in each case a renewed change in the magnetic field is carried out.

The change in the magnetic field can preferably be carried out taking into account the measured magnetic field homogeneity in that either such changes are carried out which increase the homogeneity of the magnetic field or a change is carried out which, as a result of the already carried out cycles of metrologically tracked changes in magnetic field and the analyses carried out with regard to these changes in magnetic field of changes in the heart rate variability of a test subject, leads one to expect a desired change in the heart rate variability. It has been shown that the majority of test subjects respond positively to a homogeneous magnetic field. However, there can also be cases in which a positive effect, as determined by the changes in the heart rate variability, is achieved in the case of non-homogeneous magnetic fields. In such a case, as in the case of experimentally evoked changes in magnetic field, one can call on analyses of previous changes and measurements in order to influence the magnetic field acting on the test subject in any desired direction, for example, by installing devices for producing corresponding magnetic fields in the case of the test subject, for example, his workplace.

The analysis of the heart rate variability can be carried out depending on the question over various periods of time, for example, before and after the change in magnetic field individually for in each case between 2 min and 48 h, or preferably before and/or after the change in magnetic field for 3 and/or 5 min (short-term measurement).

The analysis of the heart rate variability can preferably be carried out before and/or after the change in magnetic field over a period of 10 to 30 h (long-term measurement).

Standard values for carrying out the HRV measurement are 5 min and 24 h.

In certain embodiments of the invention, a second analysis of the heart rate variability is carried out after the change in magnetic field after 1 to 6 weeks in order e.g. to also be able to detect longer term effects of the change in magnetic field for the test subject.

Numerous methods known to experts are available for changing magnetic fields acting on test subjects. The change in the magnetic field is preferably carried out by means of switching on and off of devices which emit electromagnetic waves, the spatial displacement of devices which emit electronic or radio frequency radiation in/out of the immediate vicinity of the measurement field, positioning or removing permanent magnets in/out of the magnetic field, and/or introduction or removal of screening devices around the test subject or around electromagnetic radiation sources.

Screening devices comprise e.g. metallic or metallised films, plates, non-woven fabrics or materials which already suppress the inward radiation of electromagnetic waves. Permanent magnets do not influence the oscillation of the magnetic field as such (when an oscillating magnetic field is studied), but can bring about a displacement in the amplitudes.

The use according to the invention of the analysis of the heart rate variability has numerous advantages. The measurement method takes account of the non-linearity and complexity of the human organism. Knowledge of the self-organisation of organisms is likewise contained therein such as chaotic or fractal phenomena. Improvements or deteriorations in a dynamic system, such as the animal organism represents, can be easily quantified.

This complex requirement is currently only satisfied by the HRV measurement.

The reactions of the body to changes in the homogeneity of the ULF field come about immediately, usually within seconds to minutes.

The HRV measurement method satisfies the need for detecting changes in the human regulation system immediately and directly (i.e. in real time).

The HRV measurement method is able to detect the smallest of changes in the regulation system of the animal body.

The measurement is purely technical and is not influenced by the operator. The operator is not part of the measurement system.

Energy and information medicine-based measurement methods (bioresonance, median-based measurement methods, etc.) are able to record small changes in the body, but they are usually dependent on the involvement of the operator in the measurement itself, e.g. by actuation of a measurement stylus and are also usually dependent on his/her skill and experience.

HRV measurement is a recognised and well-understood method in other fields of the study of influence variables on physiological condition.

The HRV measurement method represents a standardised technical medical method. The HRV measurement is stored with task force parameters which are valid worldwide. (Task Force 1996).

The invention can be used in a variety of fields. Use in the field of building health, where a possible influence by magnetic fields on occupants should be minimised, is equally possible as in the scientific sector in order to study the influence of magnetic fields which are changed in a targeted manner spatially/temporally on test animals.

The invention is supposed to be explained below with reference to several examples which illustrate partial aspects, wherein reference is made to the enclosed drawings in which the following is shown:

### EXAMPLE 1

### Analysis of the Heart Rate Variability

The method used in the invention for measuring the heart rate variability should initially be described with reference to a concrete example. The use proposed by the invention of the HRV method has numerous advantages (see above) which prove the usefulness of the use of HRV analysis for determining the influence of changes in the magnetic field.

The analysis of the heart rate variability (HRV) is a quantitative method for characterising the autonomic nervous regulation processes of test subjects such as mammals.

In order to define binding measurement standards and develop physiological and pathophysiological correlations, in 1996, the European Society of Cardiology and the North American Society of Pacing and Electrophysiology founded a Task Force (Task Force 1996), on the definitions and parameters of which the current measurement standard is based, and was to a certain extent already developed further and supplemented.

In the case of HRV, the time intervals from one heart beat to the other are measured with great precision by means of ECG. Several values are then calculated with different mathematical operations from the time variability, i.e. from the variance of the time intervals of the individual heart beats, which values can be used for an evaluation and interpretation of the “condition” of the measured test subject.

A large variability of the rhythm points at a good regulation capacity of the organism. A rigid curve image with little variation is an indication of heart disease, age, blockages or generally a poor state of health.

In this case, it should be emphasised that the HRV, similar to the measurement of erythrocyte sedimentation rate, is indeed an unspecific but highly sensitive method which responds even to minimal changes in the biological system.

The heart beat of a mammal is, generally and simplistically speaking, regulated, on the one hand, by the sympathetic, on the other hand, by the parasympathetic nervous system. The stronger character, the increased dominance of one or the other part of this antagonistically operating system can thus be read in the HRV, wherein guidelines known to the person skilled in the art can be called on for the interpretation of the data. The HRV can thus also be considered as a measurement system for the stress level of a biological system.

It should be emphasised that the HRV firstly involves a non-invasive method and secondly involves a real-time measurement which has great advantages.

Since the sympathetic and the parasympathetic nervous system—both summarised under the term “autonomic nervous system”—are also responsible for the control and regulation of the internal organs, pathological conditions in the organs are also reflected in the results of the HRV in which—unspecificaily!—in turn e.g. an increased stress level can be read.

By means of the HRV, therefore, highly sensitively but unspecifically, on the one hand changes in the autonomic control and regulation processes of the test subject are detected and on the other hand—and this is where the great benefit in terms of preventive medicine lies—one can read out of HRV data what the autonomic control and regulation capacity of the respective biological system is and whether the system is stressed. (Very general statement: Stress and loss of energy of the overall system also mean the same for sub-units, i.e. cells, organs, etc. Malaise and illnesses arise precisely on this basis)

Stresses of all types, exhaustion of the control and regulation capacities as well as the energy loss of the overall system can already be clearly seen in the HRV even before, for example, a person perceives these stresses cognitively or physically—as has now been shown, also a great advantage in the context of electrosmog.

The short-term HRV enables the following evaluations:
Condition and regulation capacity of the vegetative nervous system
Condition of the heart
Individual stress level
Metabolic status (anabolic—catabolic)
Reaction to measures
Global fitness
Illness profiles

Long-term HRV furthermore enables the following evaluations, particularly also in the case of humans.

Generally:
Determining general state of health
Detecting sleep disorders

Sport
Training observation
Detecting energy loss
Detecting performance limits
Improving performance by optimising training methods

Stress management
Early detection of burn-out
Detection of stress-induced illnesses
Observation of general regulation capacity
Process control of the measures taken

Weight control
Targeted checking of the body regulation in the case of diets
Diet optimisation
Monitoring energy and performance condition during the diet

The following data is obtained from the results of an HRV measurement in the case of humans:

Time-Related Variables, Statistical Variables:

NN: Interval between two heart beats (normal to normal)

SDNN: Standard deviation of all NN intervals

SDNN-i: Mean value of the standard deviations of all NN intervals for all five-minute sections in the case of 24-hour recording

SDANN: Standard deviation of the mean value of the NN intervals in all five minutes of the entire recording

SDANN-i: Standard deviation of the mean normal NN interval for all five-minute sections in the case of recording of 24 hours

r-MSSD: Square root of the square mean value of the sum of all differences between adjacent NN intervals

pNN50: Percentage of the intervals with at least 50 ms deviation from the preceding interval (higher values indicate increased parasympathetic activity)

SDSD: Standard deviation of the differences between adjacent NN intervals

NN50: Number of pairs of adjacent NN intervals which deviate by more than 50 ms from one another in the entire recording.

RI (Relaxation Index):
Calculation is performed from the ratio of width to height of the histogram, result is 1 numerical value, referred to as “Stress Index” (SI).

RI=1/SI

The RI is a measure for the recovery capacity of the organism.

Age-corrected standard value: 50′%

VI (Variability Index):

Evaluating the histogram in terms of its width and thus the bandwidth from lowest to highest present frequencies.

A high value indicates a large width of frequencies which allows one to conclude good variability and thus vitality. Age-corrected standard value: 50%

Geometrical Variables

HRV-Triangular-Index: Integral of the density spread (number of all INN intervals divided by the maximum (height) of the density spread)

TINN: Length of the basis of the minimum square difference of the triangular interpolation for the maximum value of the histogram of all NN intervals

Various devices for analysing the heart rate variability are used on the market. A system suitable for carrying out the invention is supplied, for example, by ProQuant Medizinische Geräte Handels GmbH, Graz, AT, under the type designation “Cardio-Test”. 3 ECG electrodes are attached in the practical performance of a short-term HRV measurement (below the left and the right armpit and on the left iliac crest).

The electrodes are connected to the HRV device by means of in each case one electrode cable.

The test object lies or sits calmly and should where possible not move or speak.

A measurement program is subsequently started on the linked. PC and the measurement process begins:

While the heart rate is recorded optionally for 3 or 5 minutes, this recording of the heart beat rhythm can be tracked on a graphic window which shows the heart rate profile.

The first started measurement is referred to as a “reference measurement” or initial measurement. It represents a starting state of a person and is stored with a date and time in a log.

If manipulations of any type are subsequently carried out which have an influence on biological processes, such as, for example, the change in magnetic field carried out according to the invention on a test subject, a further measurement can subsequently be carried out, which is referred to as a “control measurement” or subsequent measurement.

The results of the control measurement are compared by software automatically with the reference measurement. Qualitative or quantitative differences to the reference measurement are represented graphically and in figures.

The software used by way of example produces evaluation diagrams with several diagram windows which can be evaluated by the user.

The diagram window “R value” displays the sum of the individual results, with 50% corresponding to the healthy average population.

The diagram window “Change” shows the difference between the two measurements. Negative values in the sense of a deterioration are in this case displayed in red in the diagram, improvements are represented as positive values and in green. Quantitatively, the changes are indicated in %.

The diagram window “Balance” shows the degree of activation of the sympathetic nervous system (“Activation”) or parasympathetic nervous system (“Relaxation”).

A clear change is present if there is a percentage difference between two measurements, for example, between the two measurements carried out according to the invention before and after the change in the magnetic field, of at least 20% in one direction.

Lower percentage changes indicate tendencies in so far as the control measurement was not taken immediately after a measure, rather hours, days or weeks later.

Lower percentage changes in a control measurement which was carried out immediately after the reference measurement and the measure taken are clearly to be assessed as “improvement” or “deterioration”.

If the intention is to study direct biological effects of changes in magnetic field, short-term measurement is used to achieve this.

The initial condition of a test subject is ascertained by reference measurement.

A change in the magnetic field, for example, of the ULF field, is subsequently carried out.

Immediately thereafter (usually within minutes), it can be ascertained by one or more short-term measurements carried out at short consecutive time intervals whether the measure, in the organism of the studied person,
causes changes or not,
and whether these changes are to be assessed as biologically positive or negative. Whether they are thus beneficial or not.

Short-term measurement is suitable if specific questions have to be clarified.

However, long-term HRV measurement is significantly more important for a broad application.

### EXAMPLE 2

HRV Long-Term Measurement

Measurement by means of long-term HRV device enables recording of longer exposure periods.

Such a measurement may, for example, be expedient if small changes in the physiological condition of a test subject are also supposed to be detected or if it is expected that the influence of the magnetic field is smaller so that changes thereof will correspondingly produce a small change in the HRV analysis. Moreover, fluctuations which are caused by other influences than the change in magnetic field are easier to compensate by means of long-term analysis as a result of their character generally across the measurement period (for example, stress level, hunger/thirst, lack of sleep, etc.). In this case, it should be ensured that the test subject is located at the desired exposure point opposite a magnetic field over a sufficiently long period of time during the entire measurement time. It can, for example, be assumed that, in the case of measurement at a workplace, in the case of a typical working time of 8 hours, a 24-hour long-term HRV measurement will still produce results in which the influence of the change in magnetic field clearly impacts on the overall result in the case of comparison of two completed. HRV measurements.

An HRV recorder which can be used by way of example from ProQuant is approximately the same size as a matchbox (5×2×1 cm) and weighs only 25 grams. It is stuck to the chest with the help of an adhesive strip (patch). 2 electrodes for recording the pulse signal are connected to the HRV recorder and also stuck to the chest and carried for 24 hours for recording heart rate data.

The evaluation software is on a data processing unit (e.g. PC). A memory card or a different removable memory from the HRV recorder is introduced into a reading station in the computer and the evaluation is carried out automatically. As a result, one obtains graphic representations and numerical values, of which the most important for the overall evaluation just as in short-term measurement—in turn is the R-value as an expression of the overall regulation quality and the current balance of the patient.

All the measurements are saved and can be called up again and printed out at any time.

The HRV recorder is e.g. sent to a test subject (person) to be tested and he attaches the device including both electrodes according to the enclosed description. The memory is then pushed into the intended opening and the measurement is automatically started by engaging the memory.

The recorder remains on the body of the test person for 24 h, with neither everyday activities nor sleep being restricted or impaired by the device.

After 24 h, the device is removed and sent back.

The data stored in the memory is read out on the laptop.

### EXAMPLE 3

### Evaluating the Measurement Results of a 24 h Long-Term Measurement

The evaluation of the measurement results can fundamentally be carried out:
by numerical sum values of R-value and balance (see above) as an expression of overall regulation capacity over 24 h:

As in the case of short-term HRV measurement, there are also overview values here which characterise the current condition in the form of a sum value.

Changes are—just as in the short-term HRV measurement—represented as a percentage decrease or increase in the sum value.

This classification enables a rapid overview of the situation.

If more detailed diagnostic statements are desired, analysis of the curve images (see 3.1.2.) can be carried out.
By analysis of the curve images of R-value, balance, frequency distribution and power spectrum generated by the software:

The R value (regulation value) is represented as an average value of several HRV parameters (RMSSD, SDNN, VI, RI) and thus reflects the overall regulation state of the patient. The heart rate curve is also represented in each case.

The main parameter which represents the sum of variables is in turn the R value (“regulation value”) (see short-term measurement), it numerically represents the quality of the overall regulation over 24 h.

In the case of the balance which is also represented graphically (see above), the ratio between activation (sympathetic nervous system) and relaxation (parasympathetic nervous system) is represented.

A further graph finally shows the frequency distribution with the exact ratios of the individual spectral components extracted by a special algorithm from the recorded heart rate: spectral components frequency bandwidth system ratio of the ANS:

VLF (very low frequency) 0.00-0.04 Hz, hypothalamic-hypophysary axis (HFA)

LF (low frequency) 0.04-0.15 Hz, vasomotor centre

HF (high frequency) 0.15-0.4 Hz parasympathetic nervous system

High Frequency (HF) blue, Low Frequency 1 (LF1) green, Low Frequency 2 (LF2) yellow, very low Frequency (VLF) red.

The so-called power spectrum which is also represented by the software in a graph corresponds to the quantitative distribution of the individual spectral frequencies. In this case, the frequencies are plotted in Hertz (Hz)—from 0.0 to 0.40 Hertz (Hz)—for orientation. A colour representation enables interpretation of the respective frequency ratios, wherein the blue to green colour spectrum signifies no to small ratios and the yellow to red colour spectrum signifies average to high ratios of the corresponding frequency. The user can thus evaluate the temporal profile of the physiological condition of a test subject by assessing the various graphs provided by the software.

### EXAMPLE 4

### Description of an Exemplary Magnetic Field Measurement

A measurement of the vertical component of the magnetic flux density is carried out, relative to the unidirectional field and the ultra-low-frequency alternating field from 0-15 Hz.

In a software-generated evaluation graphic, the mathematical evaluation of measurement values is represented, which representation approximates a topographical map.

Interference points are expressed by deviations from the natural background (=changed homogeneity pattern). The biological stimulus strength can be determined and evaluated individually by a special mathematical evaluation for each individual measurement point.

A precision tesla meter 05/40, which can be used by way of example, from the manufacturer IIREC, Linz, AT with a measurement value deviation of max. 0.5% in the case of a vertical magnetic induction of 40 microtesla and a frequency range of 0-18 Hz is assumed below.

The device records the vertical magnetic flux density above a regularly square lattice with spacings of 10 cm on a surface of 1×1 m, on sleeping areas of 1×2 m, for laboratory measurements also 0.5×0.5 with 5 cm spacing. The values measured at the lattice points in microtesla are interpolated by means of a data analysis program and represented as a 2D diagram.

The two-dimensional evaluation graphic illustrates the direct measurement result, the distribution of the vertical magnetic flux density (in microtesla). Lines connect points with the same vertical flux density (similar to height lines). The surfaces therebetween are coloured.

The graphic shows for each measurement point the biologically effective stimulus level which is produced from inhomogeneities of the magnetic field. A unit millitesla/m² is produced by computer for this variable. A small disc appears in the illustration at each measurement point, the diameter of which is proportional to the stimulus level of the measurement point. The corresponding evaluation value is entered above it.

According to experienced values of the manufacturer, the following evaluation emerges: Stimulus level in millitesia/m² evaluation

0 to 5 slight stimulus

Above 5 to 10 strong stimulus

Above 10 very strong stimulus

The graphical result representations are followed by an individual biophysical evaluation of the field ratios.

The following are evaluated:
spatial distribution of the stimulus points or stimulus zones
the level of the stimulus points or stimulus zones

The evaluation furthermore includes:
a case-related evaluation which discusses the particular features of the respective measurement points and suspected causes of stimulus points or stimulus zones
as well as the necessity of protective measures.

Classification of the Spatial Distribution of the Stimulus Points or Stimulus Zones:

Type P (point) punctiform occurrence

Type L (line) along a straight line (“stimulus beam”)

Type A (area) superficial distribution

Classification of Places:

Type SP (sleeping place) Sleeping place

Type WP (working place) Working place

Type LP (living place) Other place where one spends time, e.g. living room.

If the method is supposed to be used to improve the physiological condition of test subjects, it is necessary to make changes in the magnetic field as a result of the type of place and the maximum level of the stimulus points or stimulus zones. It is classified according to a generally common scale as follows:

S (small): Measures in the case of particular sensitivity

M (medium): Measures recommended

L (large): Measures urgently required

XL (extra large): Measures very urgently required

XXL: Measures very urgently demanded

Practical Performance of the Measurement:

1. Setting Up the Measurement Grid

Sizes: 1×1 m for seats, 2×1 m for sleeping places

The measurement grid is clamped a few cm above the lying area of the person in the case of beds or placed directly on the mattress.

In the case of workplaces or other places, the measurement grid is adjusted to the chest height of the person who is normally located in this place.

2. Image Documentation

Photo of the measurement grid set up at the location in order to produce the same situation in the case of a subsequent measurement.

3. Measurement

The entire measurement surface defined by the measurement grid is measured in the grid of 10 cm with the precision tesla meter. The measurement value of each measured point is entered into the measurement software on the laptop.

4. Evaluation of the Measurement Data:

The measurement data is sent via the Internet to the evaluation portal, as a result one once again obtains via the Internet a complete measurement log (see above) including brief classifications of the measured place in terms of its biological quality.

### EXAMPLE 5

Linking and iteration of the results of biological measurement (HRV) in human beings and physical measurement of the ULF field (using the HRV measurement system from ProQuant).

The system used by way of example from ProQuant indicates a sum value (“R value”=regulation value) as an additional parameter which other manufacturers do not offer. This enables a very simple overall statement.

The R value expresses the following:

It acts as a mean value of variables and correlates with the calculation of the “Total Power”. The latter is in turn used very frequently worldwide in evaluation as one of the main parameters.

The relationship between the results of both measurement methods—on the ULF field and on humans—is very simple to establish:

An improvement in the homogeneity of the ULF field results in an improvement in the regulation capacity and vitality of human beings.

It is the same case vice versa: low homogeneity of the ULF field stresses human beings and leads to reduction of regulation capacity and vitality.

The following classification scheme must be summarised:

A. Evaluation Diagram of the FCM/FGD Measurement:

S (small): Measures in the case of particular sensitivity

M (medium): Measures recommended

L (large): Measures urgently required.

XXL: Measures very urgently demanded

B. Evaluation Result of the HRV Measurement

In the form of the R value, numerically 0-100

| FCM | HRV, R value | Recommendation for action |
|---|---|---|
| S | <50 | Good vitality, no changes in |
| M | | magnetic field required. |
| | | HRV check (2^{nd} |
| | | measurement) not required |
| | 40-50 | Slight reduction in vitality, |
| | | changes in magnetic field |
| | | preventively possible. HRV |
| | | check not required |
| | 25-40 | More significant reduction |
| | | in vitality, changes in |
| | | magnetic field |
| | | recommended, HRV check |
| | <25 | Significant restriction in |
| | | vitality, medical |
| | | clarification recommended |
| | | if the measurement was not |
| | | carried out after significant |
| | | stress (sport). Subsequently |
| | | changes in magnetic field. |
| | | HRV check |
| | >50 | Good vitality, changes in |
| | | magnetic field preventively |
| | | recommended, HRV check |
| | | not required |
| | 40-50 | Slight reduction in vitality, |
| | | changes in magnetic field |
| | | preventively expedient. |
| | | HRV check recommended |
| | | in ½ year |
| | 25-40 | More significant restriction |
| | | in vitality, changes in |
| | | magnetic field |
| | | recommended, HRV check |
| | <25 | Significant restriction in |
| | | vitality, medical |
| | | clarification recommended |
| | | if the measurement was not |
| | | carried out after significant |
| | | stress (sport). Subsequently |
| | | changes in magnetic field. |
| | | HRV check |
| L | >50 | Good vitality, changes in |
| | | magnetic field preventively |
| | | recommended. HRV check |
| | | not absolutely necessary |
| | 40-50 | Slight reduction in vitality, |
| | | changes in magnetic field |
| | | recommended. HRV check |
| | | recommended. |
| | 25-40 | More significant restriction |
| | | in vitality, changes in |
| | | magnetic field urgently |
| | | recommended, HRV check |
| | <25 | Significant restriction in |
| | | vitality, medical |
| | | clarification recommended |
| | | if the measurement was not |
| | | carried out after significant |
| | | stress (sport). Subsequently |
| | | changes in magnetic field. |
| | | HRV check |
| XL | >50 | Good vitality, changes in |
| | | magnetic field as a result of |
| | | field measurement still |
| | | recommended, HRV check |
| | | recommended in ½ year |
| | 40-50 | Slight reduction in vitality, |
| | | changes in magnetic field |
| | | recommended, HRV check |
| | | recommended in 4 months |
| | 25-40 | More significant restriction |
| | | in vitality, changes in |
| | | magnetic field urgently |
| | | recommended. HRV check |
| | <25 | Significant restriction in |
| | | vitality, medical |
| | | clarification recommended |
| | | if the measurement was not |
| | | carried out after significant |
| | | stress (e.g. intensive sport). |
| | | Subsequently changes in |
| | | magnetic field urgently |
| | | required. HRV check |
| | | urgently required |
| XXL | >50 | Good vitality, changes in |
| XXL | | magnetic field as a result of |
| | | field measurement still |
| | | recommended, HRV check |
| | | recommended in ½ year |
| | 40-50 | Slight reduction in vitality, |
| | | changes in magnetic field as |
| | | a result of field |
| | | measurement still urgently |
| | | recommended, HRV check |
| | | recommended in 3 months |
| | 25-40 | More significant restriction |
| | | in vitality, changes in |
| | | magnetic field urgently |
| | | demanded, HRV check |
| | <25 | Significant restriction in |
| | | vitality, medical |
| | | clarification recommended |
| | | if the measurement was not |
| | | carried out after significant |
| | | stress (e.g. intensive sport). |
| | | Subsequently changes in |
| | | magnetic field urgently |
| | | demanded. HRV check |

Procedural Steps:

1. The person whose workplace or sleeping place is supposed to be measured is first sent an HRV recorder (see above) by post.

2. The test person attaches the measurement device including electrodes in accordance with the enclosed description, starts the measurement by pushing in the memory module, and removes the device again after 24 h. The measurement is automatically terminated by removal of the memory. The test person subsequently returns the device and memory.

3. The HRV measurement is evaluated with the normal evaluation software.

4. A technician in measurement technology comes to the location and performs the magnetic field measurement. The measurement is also evaluated.

5. The results of the two measurements are combined by means of special software. In accordance with the above iteration diagram, the technician in measurement technology receives an overall evaluation of the two measurements and the further recommended/necessary/unnecessary steps are specified automatically in a written form.

The above iteration diagram was related to the HRV device type from ProQuant. In a similar form, this diagram can also be adapted to devices from other manufacturers.

### EXAMPLE 6

### Practical Procedure on the Basis of a Sleeping Place

In the following example, for demonstration purposes, the HRV measurement is combined with a magnetic field measurement according to Example 4 and an evaluation as described in Example 5 is used.

The test subject, a German businessman, had sleep disorders, reported stress symptoms and burn-out states; suffered from concentration disorders and recurring urinary tract infections. He blamed this stress subjectively on his work-related stress.

In the example, due to the sleep disorders described, the sleeping place and not the workplace was measured first.

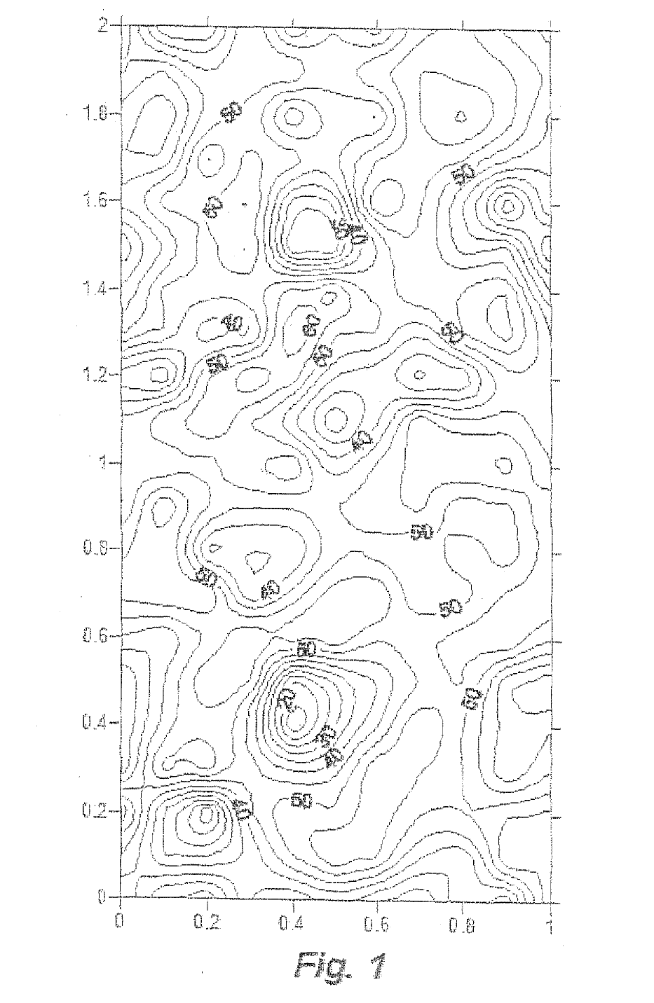

A measurement carried out in accordance with Example 4 resulted in the magnetic field situation shown in FIG. 1. FIG. 1 illustrates in this case the direct measurement result as a distribution of the vertical flux density in mT. The lines connect points with the same vertical flux density. The surfaces located therebetween have a different coloured background which is reproduced as shades of grey. The coordinates are length in m.

The normal value is approx. 42 mT in Central Europe. In the example shown of FIG. 1, the measurement values lie between 10 and 80 mT which already indicates a significant inhomogeneity of the magnetic field at the sleeping place.

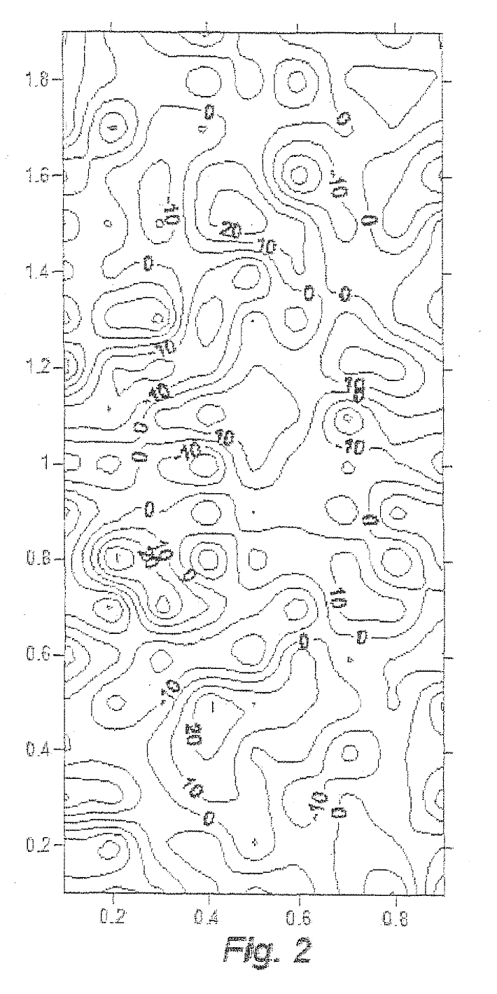

FIG. 2 shows the result of a mathematical evaluation by means of the evaluation software supplied with the device. It shows for each measurement point the biologically effective level of stimulus which is produced from the inhomogeneities of the measurement field. This variable has the unit [mT/m²]. The following measurement result is found at the sleeping place measured by way of example:

Level of the stimulus points: the maximum amount is 47.95 mT/m² at the coordinate points [0.2; 0.8].

The case-related evaluation produces a large number of stimulus point distributed across the entire measurement field.

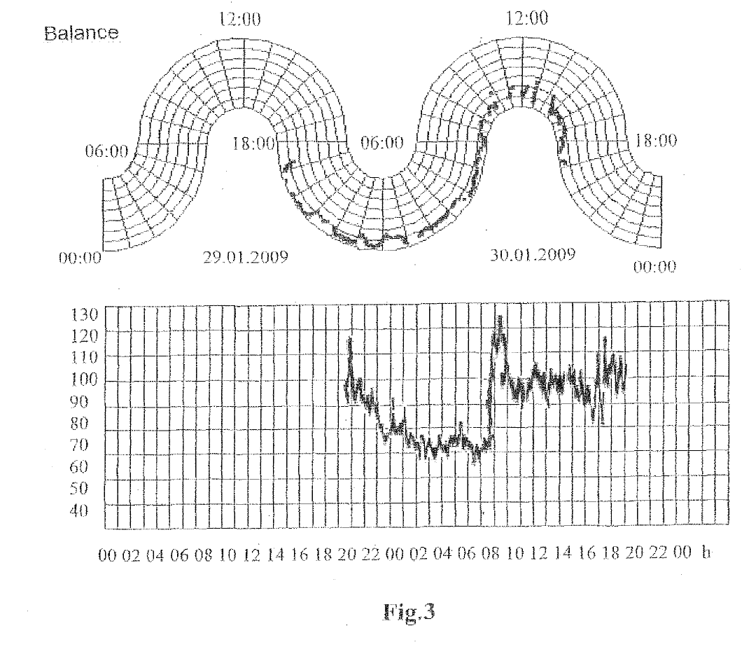

The first HRV long-term measurement carried out according to the invention on the test subject produces the result shown in FIG. 3 that shows the “balance” of the measurement. Both the measurement values of the measurements carried out over the day and the night-time measurements show that the test subject is also exposed to stress during the night, when the parasympathetic nervous system should actually be more active, with the result that no sleep regeneration takes place.

The magnetic field at the sleeping place was deliberately changed after the HRV analysis by various measures:
Replacement of the spring core mattress with a metal-free model;
Removal of electronic devices from the close vicinity of the bed;
Removal of the (metal-coated) mirror;
Fitting of magnetic field-active films to remaining metal parts; including a transformer and the slatted frame of the bed.

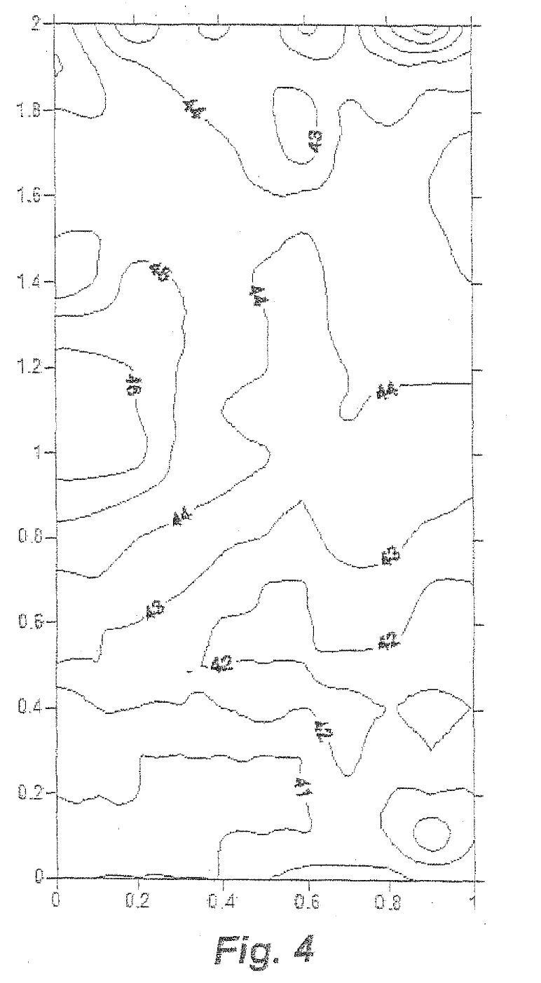

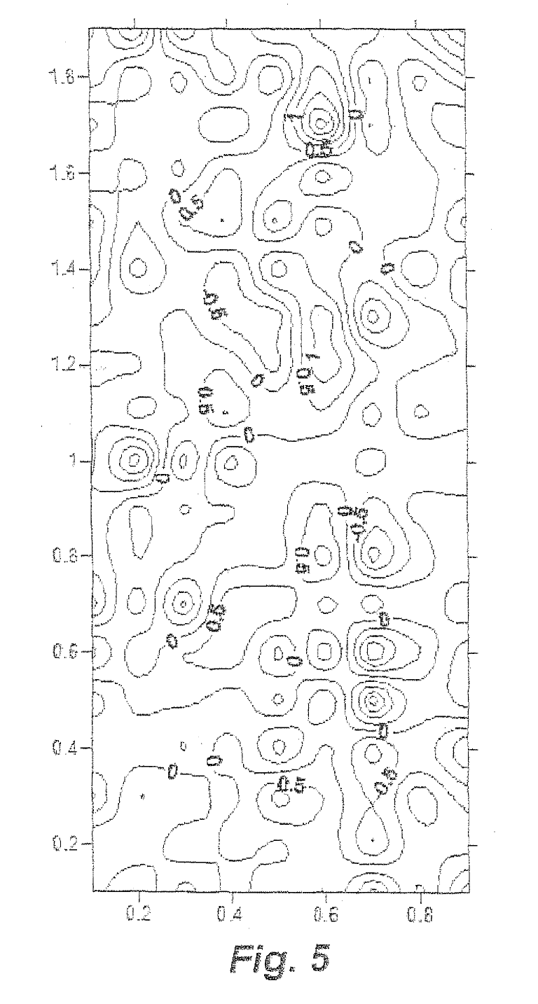

A further magnetic field measurement and a further HRV measurement were carried out. The results of the magnetic field measurement are shown in FIGS. 4 and 5. The distribution of the vertical magnetic flux density now exhibited values between 38 and 47 mT and thus significantly lower inhomogeneities as is also apparent from FIG. 5. The level of the stimulus points changes to a maximum of 3 mT/m² at the coordinates [0.6; 1.7].

The case-related evaluation shows that the intensity of the stimulus points has fallen significantly and at 3 mT/m² only corresponds to weak stimuli.

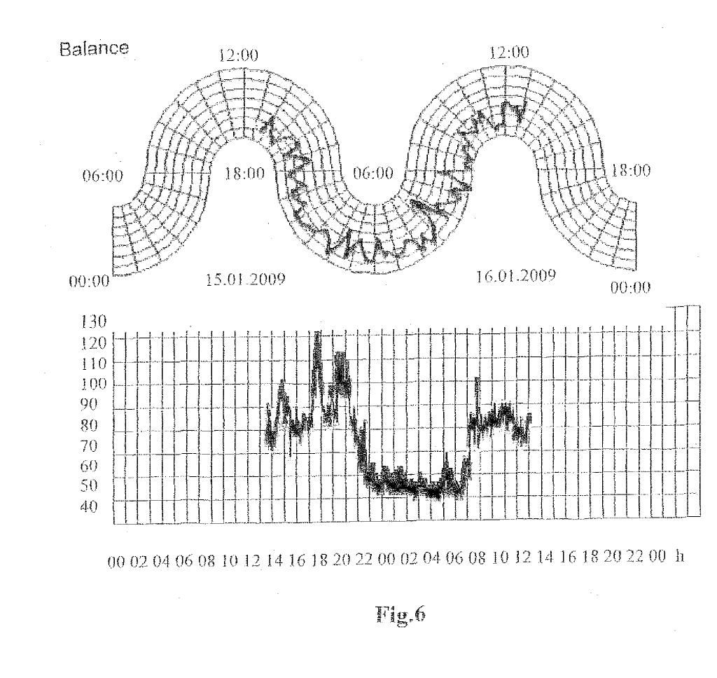

The second HRV analysis according to the invention produced the result shown in FIG. 6 for the R value. The curve has moved significantly, the physiological condition is significantly better, apparent in the fact that the R value has increased in comparison to the first measurement from 32% to 66%.

These results coincide with the subjective impressions of the test subject who reports improved sleep and less stress.

The use according to the invention of HRV analyses for determining the physiological condition of a test subject before and after a change in magnetic field thus clearly shows the effects of the change in magnetic field on the organism of the test subject.

It should be noted that, in this case, for control purposes, both the HRV measurement and the magnetic field measurement were carried out, but the magnetic field measurement is optional since the HRV measurement alone was able to shown the physiological changes as a result of the change in magnetic field on the test subject.

1. A method of using a device for analysing heart rate variability in order to determine changes in the physiological condition of a test subject due to a change in a magnetic field acting on the test subject, comprising the analysis of the heart rate variability of the test subject in each case before and after the change in the acting magnetic field.

2. The method according to claim 1, wherein the test subject is a mammal.

3. The method according to claim 2, wherein the mammal is a human.

4. The method according to claim 1, wherein the measurements and the changes in magnetic field are carried out in immediate succession.

5. The method according to claim 1, wherein the renewed analysis of the heart rate variability is carried out 1 to 30 days after the change in the magnetic field.

6. The method according to claim 1, wherein the analysis of the heart rate variability comprises:
measuring the pulse of the test subject by means of an ECG;
determining the heart rate variability from the pulse; and
evaluating the heart rate variability in terms of the physiological condition of the test subject.

7. The method according to claim 6, wherein the analysis furthermore includes the generation of a regulation value (R value) which numerically reflects the quality of the physiological condition of the test subject over the period of measurement.

8. The method according to claim 7, wherein the presence of a change in the physiological condition of the test subject is assumed in the event of a change by more than 10%, preferably by more than 20% in the case of the R value.

9. The method according to claim 1, further comprising the use of a device for measuring the magnetic field acting on the test subject, for correlation of the change in the magnetic field with the change in the physiological condition of the test subject.

10. The method according to claim 9, wherein the measurement is carried out in a frequency range from 0 to 15 Hz of oscillating or fluctuating magnetic fields.

11. The method according to claim 9, wherein the measurement is carried out on one plane at a spatial position at which the test subject spends at least some time during analysis of the heart rate variability, the measurement having the following steps.
definition of a surface, which lies on the plane, of a predefined size;
specifying a pattern of measurement points on the surface;
measuring the magnetic field strength at the measurement points; and
determining the magnetic field and the magnetic field homogeneity across the measured surface.

12. The method according to claim 9, wherein the change in the magnetic field is carried out taking into account a measured magnetic field homogeneity in that either such changes are carried out which increase the homogeneity of the magnetic field or a change is carried out which, as a result of the already carried out cycles of metrologically tracked changes in magnetic field and the analyses carried out with regard to these changes in magnetic field of changes in the heart rate variability of a test subject, leads one to expect a desired change in the heart rate variability.

13. The method according to claim 1, wherein it is applied several times, wherein in each case a renewed change in the magnetic field is carried out.

14. The method according to claim 1, wherein the analysis of the heart rate variability is carried out before and after the change in magnetic field individually for in each case between 2 min and 48 h.

15. The method according to claim 14, wherein the analysis of the heart rate variability is carried out before and/or after the change in magnetic field for 3 or 5 min.

16. The method according to claim 14, wherein the analysis of the heart rate variability is carried out before and/or after the change in magnetic field for between 10 and 30 h.

17. The method according to claim 1, wherein a further analysis of the heart rate variability is carried out after the change in magnetic field after 1 to 6 weeks.

18. The method according to claim 1, wherein the change in the magnetic field is carried out by means of switching on and off of devices which emit electromagnetic waves, the displacement of electronic devices or devices which emit radio frequency radiation, positioning or removing permanent magnets in/out of the magnetic field, and/or introduction or removal of screening devices around the test subject and/or electromagnetic radiation sources.

19. Method for determining changes in the physiological condition of a test subject on the basis of his heart rate variability due to a change in a magnetic field acting on the test subject, comprising the steps:
analysing the heart rate variability of the test subject;
carrying out changes to the magnetic field acting on the test subject;
renewed analysis of the heart rate variability of the test subject, and
evaluating a change in the physiological condition of the test subject on the basis of the change in the heart rate variability between the measurements before and after the change in the magnetic field.

20. Method according to claim 19, wherein the test subject is a mammal.

21. Method according to claim 20, wherein the mammal is a human.

22. Method according to claim 19, wherein the steps are carried out in immediate succession.

23. Method according to claim 19, wherein the renewed analysis of the heart rate variability is carried out 1 to 30 days after the change in the magnetic field.

24. Method according to claim 19, characterised in that the analysis of the heart rate variability comprises:
measuring the pulse of the test subject by means of an ECG;
determining the heart rate variability from the pulse; and
evaluating the heart rate variability in terms of the physiological condition of the test subject.

25. Method according to claim 24, wherein the analysis furthermore includes the generation of a regulation value (R value) which numerically reflects the quality of the physiological condition of the test subject over the period of measurement.

26. Method according to claim 25, wherein the presence of a change in the physiological condition of the test subject is assumed in the event of a change by more than 10%, preferably by more than 20% in the case of the R value.

27. Method according to claim 19, further comprising the use of a device for measuring the magnetic field acting on the test subject, for correlation of the change in the magnetic field with the change in the physiological condition of the test subject.

28. Method according to claim 27, wherein the measurement is carried out in a frequency range from 0 to 15 Hz of oscillating or fluctuating magnetic fields.

29. Method according to claim 27, wherein the measurement is carried out on one plane at a spatial position at which the test subject spends at least some time during analysis of the heart rate variability, the measurement having the following steps.
definition of a surface, which lies on the plane, of a predefined size;
specifying a pattern of measurement points on the surface;
measuring the magnetic field strength at the measurement points; and
determining the magnetic field and the magnetic field homogeneity across the measured surface.

30. Method according to claim 19, wherein the method is repeated several times, wherein in each case a renewed change in the magnetic field is carried out.

31. Method according to claim 19, wherein the change in the magnetic field is carried out taking into account the measured magnetic field homogeneity in that either such changes are carried out which increase the homogeneity of the magnetic field or a change is carried out which, as a result of the already carried out cycles of metrologically tracked changes in magnetic field and the analyses carried out with regard to these changes in magnetic field of changes in the heart rate variability of a test subject, leads one to expect a desired change in the heart rate variability.

32. Method according to claim 19, wherein the analysis of the heart rate variability is carried out before and after the change in magnetic field individually for in each case between 2 min and 48 h.

33. Method according to claim 32, wherein the analysis of the heart rate variability is carried out before and/or after the change in magnetic field for 3 and/or 5 min.

34. Method according to claim 32, wherein the analysis of the heart rate variability is carried out before and/or after the change in magnetic field for between 10 and 30 h.

35. Method according to claim 19, a further analysis of the heart rate variability is carried out after the change in magnetic field after 1 to 6 weeks.

36. Method according to claim 19, wherein the change in the magnetic field is carried out by means of switching on and off of devices which emit electromagnetic waves, the displacement of electronic devices or devices which emit radio frequency radiation, positioning or removing permanent magnets in/out of the magnetic field, and/or introduction or removal of screening devices around the test subject and/or around electromagnetic radiation sources.
